(19) United States
(12) Patent Application Publication
(10) Pub. No.: US 2010/0065066 A1
(43) Pub. Date: Mar. 18, 2010

(54) ORAL APPLIANCE

(75) Inventor: Richard D. Hamburg, Setauket, NY (US)

Correspondence Address: THE FARRELL LAW FIRM, LLP, 290 Broadhollow Road, Suite 210E, Melville, NY, 11747, US

(73) Assignee: RH ASSOCIATES LLC, Setauket, NY (US)

(21) Appl. No.: 12/233,308

(22) Filed: Sep. 18, 2008

(51) Int. Cl. A61F 5/56

(52) U.S. Cl. 128/848

(57) ABSTRACT

An oral appliance is provided that controls positioning of a patient's jaw. A first dental encasing component is formed to overlay at least a portion of the patient's upper teeth and has a lingual surface, a labial surface, and at least one ledge extending along the labial surface of the first dental encasing component. A second dental encasing component is formed to overlay at least a portion of the patient's lower teeth and has a lingual surface and a labial surface. At least one rotating body is rotatably fixed to the labial surface of the second dental encasing component. The at least one rotating body and the at least one ledge are disposed in positions on their respective labial surfaces to enable the at least one rotating body to contact and rotate along a length of the at least one ledge as the patient's mouth closes, advancing the patient's jaw forward.

### BACKGROUND OF THE INVENTION

1. Field of the Invention

The present invention relates to an oral appliance, and more particularly, to an improved oral appliance for prevention of snoring and obstructive sleep apnea.

2. Description of the Related Art

Snoring and obstructive sleep apnea occur when there is a partial occlusion in a patient's airway, typically involving the patient's tongue. By advancing the patient's lower jaw, the tongue is carried forward and the tongue's blocking of the patient's airway is reduced. The unblocked airway allows for increased airflow, which decreases snoring and prevents obstructive sleep apnea.

Removable appliances for use in mandibular advancement during sleep are known in the art. The mandibular advancement appliances are typically worn by patients in accordance with instructions from a doctor, dentist or orthodontist. Such appliances are constructed for use on both upper and lower dental arches. Both the upper and lower dental arches include respective engaging surfaces that set the patient's mouth in a position in which the lower jaw is advanced. The respective surfaces may attach the dental arches to each other preventing movement of the jaw during sleep. This attachment may also hold the patient's mouth open to allow for increased airflow during sleep. Non-attached engagements have also been shown in which the surfaces project upwardly from a lower arch and downwardly from an upper arch. The upwardly extending projections are engaged in front of the downwardly extending projections allowing the jaw to open and close while still being held in an advanced position.

When non-attached engagements are used, the rate of mandibular advancement is determined by the angle of the projecting surfaces. Once the surfaces are in contact, the mandibular segment continues to advance forward at a fixed rate until it reaches a maximum position. When the maximum setting is too close to the patient's maximum mandibular protrusion, tension on the Temporal Mandibular Joint (TMJ) can be severe. The only way to relieve the tension is to open the mouth, thereby decreasing the advancement and negating any improvement in airway obstruction.

The exact positioning and dimensions of the engaging surfaces are determined when an impression is taken of the patient's teeth. However, due to errors in measuring the patient's centric and maximum protrusion position, or due to errors in dental lab manufacturing, the engaging surfaces may be improperly positioned. This may result in an appliance that causes the patient's jaw to protrude too far, or an appliance that does not provide adequate advancement to compensate for the airway obstruction. A screw mechanism may be utilized to provide adjustments; however, it has a finite expansion.

A need exists for an appliance that improves the manner in which the mandible is advanced, while also allowing for mandibular advancement adjustments and means for integration with separate elements that assist in the prevention of snoring and obstructive sleep apnea.

### SUMMARY OF THE INVENTION

The present invention has been made to address at least the above problems and/or disadvantages and to provide at least the advantages described below. Accordingly, an aspect of the present invention provides an oral appliance that controls mandibular advancement.

Another aspect of the present invention provides an oral appliance having an adaptor that guides air or oxygen into the mouth of the patient.

A further aspect of the invention provides a tubing adaptor for attachment to an oral appliance.

According to one aspect of the present invention, an oral appliance is provided that controls positioning of a patient's jaw. A first dental encasing component is formed to overlay at least a portion of the patient's upper teeth and has a lingual surface, a labial surface, and at least one ledge extending along the labial surface of the first dental encasing component. A second dental encasing component is formed to overlay at least a portion of the patient's lower teeth and has a lingual surface and a labial surface. At least one rotating body is rotatably fixed to the labial surface of the second dental encasing component. The at least one rotating body and the at least one ledge are disposed in positions on their respective labial surfaces to enable the at least one rotating body to contact and rotate along a length of the at least one ledge as the patient's mouth closes, advancing the patient's jaw forward.

According to another aspect of the present invention, an oral appliance is provided. A dental encasing component is formed to overlay at least a portion of the patient's upper teeth and has a lingual surface and a labial surface. A tubing adaptor is disposed at an anterior portion of the labial surface of the dental encasing component for attachment of a tube and guidance of air or oxygen into a patient's mouth.

According to a further aspect of the present invention a tubing adaptor is provided. The tubing adaptor has one or more open projections for detachably engaging at least one aperture in an anterior portion of a dental encasing component, and for guiding air through the dental encasing component. The tubing adaptor further has at least one groove for detachably engaging at least one tube and guiding air or oxygen from the tube, through the tubing adaptor, and to the dental encasing component.

### BRIEF DESCRIPTION OF THE DRAWINGS

Aspects, features and advantages of the present invention will become more apparent from the following detailed description when taken in conjunction with the accompanying drawings in which:

FIGS. 1a and 1b are diagrams illustrating a side view of separated upper and lower dental arches of an oral appliance, according to an embodiment of the present invention;

FIG. 2 is a diagram illustrating a side view of upper and lower dental arches of an oral appliance, according to an embodiment of the present invention; and

FIG. 3 is a diagram illustrating a front view of upper and lower dental arches of an oral appliance, according to an embodiment of the present invention.

### DETAILED DESCRIPTION OF THE PREFERRED EMBODIMENTS

Preferred embodiments of the present invention are described in detail with reference to the accompanying drawings. In the drawings, the same or similar elements are denoted by the same or similar reference numerals even though they are depicted in different drawings. Detailed descriptions of constructions or processes known in the art may be omitted to avoid obscuring the subject matter of the present invention.

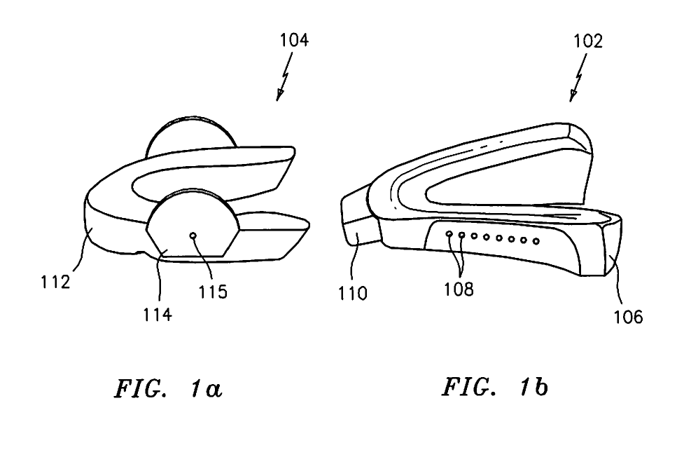

Referring initially to FIGS. 1a and 1b, the diagrams illustrate a side view of separated upper and lower dental arches, according to an embodiment of the present invention. An upper dental arch 102 and a lower dental arch 104 are formed to overlay the patient's upper and lower teeth, respectively. In the embodiment shown in FIG. 1, the upper dental arch 102 includes a first dental encasing component 106 that encases all the upper teeth of the patient. Alternative embodiments may encase as many teeth as necessary for the oral appliance to be held in place and still operate effectively. The first dental encasing component 106 includes a lingual surface and a labial surface. The lingual surface is an interior wall of the first dental encasing component that covers at least the interior side of the teeth next to the patient's tongue. The labial surface is an exterior wall of the first dental encasing component that covers at least the exterior side of the teeth next to the patient's lips.

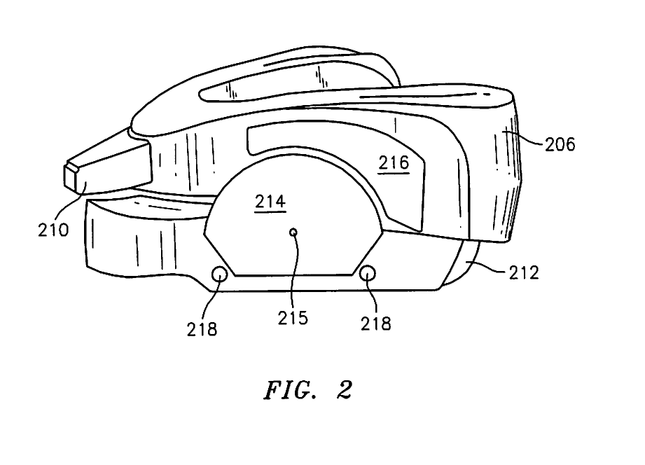

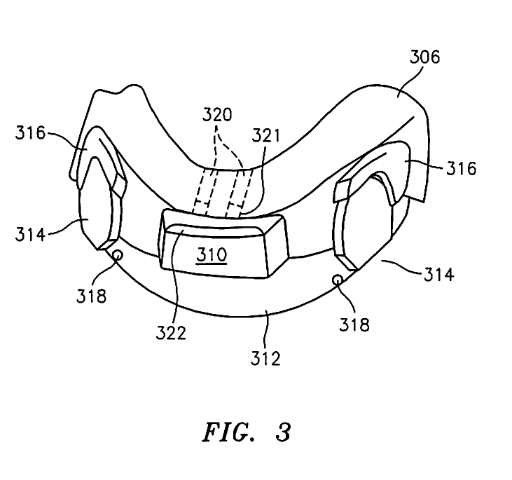

The first dental encasing component 106 has a plurality of apertures 108 running along an anterior-posterior direction on one or both labial sides of the first dental encasing component 106. A ledge component, which is described in greater detail in FIGS. 2 and 3, may be detachably fixed to the first dental encasing component 106, using at least one of the apertures 108. The plurality of apertures 108 allows the ledge component to be disposed in a plurality of different positions along a side of the first dental encasing component 106. More specifically, the plurality of apertures allow for versatility in treating a patient because the ledge may be positioned according to the needs of a specific patient, and may be changed during a patient's treatment.

The upper dental arch 102 of FIG. 1 is also shown with a tubing adaptor 110 that may be disposed at the front, or anterior portion, of the first dental encasing component 106. The tubing adaptor 110 holds a tube in place and provides a means to guide air or oxygen through the upper dental arch 102 and into the patient's mouth, allowing for further improvement in the treatment of snoring and obstructive sleep apnea.

The tube may be from an oral pressure appliance, which uses pressure to reduce an airway obstruction. The guidance of increased air or oxygen into the mouth quickly satisfies a demand for more air or oxygen caused by a more open airway, and reduces further airway collapse which can be caused by the suction forces of the lungs. The oxygen or air from the oral pressure appliance is typically passed from a tank through a relatively larger tube into a narrower tube that fits into the tubing adaptor 110. The size of the airway opening relative to tubing results in an increase in velocity of air or oxygen, jettisoning it into the lungs by virtue of the Venturi Effect.

The tubing adaptor 110 may be integrated into the first dental encasing component 106, or may be detachably fixed to the first dental encasing component 106. When the tubing adaptor 110 is detachably fixed to the first dental encasing component 106, the upper dental arch 102 may be utilized with or without the tubing adaptor 110 attached.

The lower dental arch 104 includes a second dental encasing component 112 that is formed to encase the patient's lower teeth. As described above, alternative embodiments may encase as many teeth as necessary for the oral appliance to be held in place and still operate effectively. A rotating body 114 is rotatably fixed on both sides of the second dental encasing component 112. The rotating body 114 rotates around a pivot point 115 attached to the second dental encasing component 112. The rotating body 114 has a curved upper portion and a flat lower portion. This shape allows the rotating body 114 to extend upwardly beyond the lower teeth of the patient while not extending lower than the second dental encasing component 112. Further downward extension of the rotating body 114 would cause discomfort to the gums of the patient.

FIG. 2 illustrates a side view of upper and lower dental arches of an oral appliance, according to an embodiment of the present invention. A first dental encasing component 206 is shown having a tubing adaptor 210 disposed at a front anterior portion of the first dental encasing component 206, and a ledge 216 disposed on a side of the first dental encasing component 206. An additional ledge may be disposed on an opposing side of the first dental encasing component 206. The ledge 216 may be integrated into the first dental encasing component 206 or the ledge 216 may be a separate attachment. When the ledge 216 is a separate attachment, it may be detachably fixed to the upper dental arch through the use of apertures 108 shown in FIG. 1. Further, ledge 216 may be positioned and adjusted in an anterior or posterior direction according to the needs of the patient. The ledge 216 has a curved surface extending from an upper, more anterior portion of the first dental encasing component 206 to a lower, more posterior portion of the first dental encasing component 206. In an embodiment of the present invention, the ledge 216 has an integrated groove that allows for engagement with another surface.

A second dental encasing component 212 is shown having a rotating body 214 rotatably fixed at a pivot point 215 to a side of the second dental encasing component 212. The second dental encasing component 212 also has a pair of projections 218 that are positioned to limit the rotation of the rotating body 214. An additional rotating body 214 and pair of projections 218 may be disposed on an opposing side of the second dental encasing component 212. The rotating body 214 is disposed for engagement with the ledge 216 of the upper dental arch 206. More specifically, as a patient's mouth closes, the rotating body 214 rotates along the curved surface of ledge 216, advancing the mandible forward. This rotation along the surface of the ledge provides additional ease in advancement. The projections 218 limit the rotation of the rotating body 214 in accordance with the patient's needs. Further, the projections 218 prevent rotation of the rotating body 214 into the lower gums of the patient. In an embodiment of the present invention, the surface of the rotating body 214 also engages the integrated groove in the ledge 216 so that the rotating body 214 may not slip laterally off the ledge 216 during rotation.

The ledge 216 and the rotating body 214 are both specifically shaped in accordance with the needs of the patient. For example, the ledge 216 and the rotating body 214 are shaped with a curvature that follows a smooth and natural mouth closing motion of the patient while still advancing the mandible forward. The guided closure of the patient's mouth is also affected by the limited rotation of the rotating body 214 according to where the projections 218 are set. More specifically, the rotating body 214 allows for movement of the jaw while maintaining the necessary protrusion of the mandible. The steepness of the degree of advancement as well as the amount of maximum mandibular protrusion are controlled by the changing curvature and position of the rotating body 214. Further, the rate of mandibular advancement may start slowly, then increase toward a mid-position, then slow the rate of advancement at the end of the mandible's maximum protrusion. This places less stress on the TMJ. More customization is possible to further reduce tension on the TMJ by transcribing the patient's jaw motion to the shape of the rotating body 214. This offers the least stress on the TMJ, while advancing the jaw to its appropriate position.

FIG. 3 illustrates a front view of upper and lower dental arches of an oral appliance, according to an embodiment of the present invention. A first dental encasing component 306 is shown with two ledges 316 and a tubing attachment 310. A second dental encasing component 312 is shown with two rotating bodies 314 and projections 318. The tubing attachment 310 is attached to tunnels 320 provided through the first dental encasing component 306 that allow air or oxygen to flow from a tube into the mouth of the patient. The tunnels 320 guide air or oxygen into the first dental encasing component 306, around the patient's teeth, and exit at the interior of the patient's mouth. The tubing attachment 310 is preferably attached by inserting open projections 321 of the tubing attachment 310 into the tunnels 320 of the first dental encasing component 306. Further, the tubing attachment 310 includes a groove 322 across its front in which the tube may be detachably held when air is being guided into the mouth of the patient. The front groove 322 guides air into the tubing attachment 310, out the open projections 321 and into the tunnels 320. The tubing attachment 310 may also be integrated into the first dental encasing component 306.

Thus, the embodiments of the present invention provide an appliance that improves the manner and ease of mandibular advancement through the use of a rotating body, while also providing versatility during treatment through possible adjustments. The embodiments of the present invention also provide for possible adaptation of the appliance for use with oral pressure appliances. The improved mandibular advancement and increased airflow provide a patient with greater success in eliminating snoring and sleep apnea.

While the invention has been shown and described with reference to certain preferred embodiments thereof, it will be understood by those skilled in the art that various changes in form and details may be made therein without departing from the spirit and scope of the invention.

What is claimed is:

1. An oral appliance that controls positioning of a patient's jaw comprising:
a first dental encasing component formed to overlay at least a portion of the patient's upper teeth and having a lingual surface, a labial surface, and at least one ledge extending along the labial surface of the first dental encasing component;
a second dental encasing component formed to overlay at least a portion of the patient's lower teeth and having a lingual surface and a labial surface;
at least one rotating body rotatably fixed to the labial surface of the second dental encasing component;
wherein the at least one rotating body and the at least one ledge are disposed in positions on their respective labial surfaces to enable the at least one rotating body to contact and rotate along a length of the at least one ledge as the patient's mouth closes, advancing the patient's jaw forward.

2. The oral appliance of claim 1, wherein the at least one ledge comprises a groove for engagement with a surface of the at least one rotating body.

3. The oral appliance of claim 1, wherein the at least one ledge is integrated into the labial surface of the first dental encasing component.

4. The oral appliance of claim 1, further comprising at least one ledge component detachably fixed to the first dental encasing component and comprising the at least one ledge.

5. The oral appliance of claim 4, wherein the at least one ledge component is detachably fixed one of a plurality of possible positions along the labial surface of the first dental encasing component in accordance with a patient's needs.

6. The oral appliance of claim 1, wherein the second dental encasing component comprises at least one projection extending outward from the labial surface limiting rotation of the at least one rotating body.

7. The oral appliance of claim 1, further comprising a tubing adaptor disposed at an anterior portion of the labial surface of the first dental encasing component for attachment of a tube and guidance of air or oxygen into a patient's mouth.

8. The oral appliance of claim 7, wherein the tubing adaptor is integrated into the first dental encasing component.

9. The oral appliance of claim 7, wherein the tubing adaptor is detachably fixed to the first dental encasing component.

10. The oral appliance of claim 9, wherein the first dental encasing component comprises at least one aperture in the anterior portion for accepting open projections of the tubing adaptor for attachment of the tubing adaptor, and for guiding air or oxygen through the dental encasing component.

11. The oral appliance of claim 9, wherein the tubing adaptor comprises at least one groove for detachably engaging at least one tube when guiding air or oxygen from the tube, through the tubing adaptor, and to the dental encasing component.

12. The oral appliance of claim 1, wherein the at least one ledge curves from a lower posterior portion of the labial surface of the first dental encasing component in an upper anterior direction.

13. The oral appliance of claim 1, wherein a rotating body is disposed on each posterior side of the second dental encasing component for engagement with a ledge on each posterior side of the first dental encasing component.

14. The oral appliance of claim 1, wherein the at least one rotating body comprises an upper curved surface and a lower substantially flat surface.

15. An oral appliance comprising:
a dental encasing component formed to overlay at least a portion of the patient's upper teeth and having a lingual surface and a labial surface; and
a tubing adaptor disposed at an anterior portion of the labial surface of the dental encasing component for attachment of at least one tube and guidance of air or oxygen into a patient's mouth.

16. The oral appliance of claim 15, wherein the tubing adaptor is integrated into the dental encasing component.

17. The oral appliance of claim 15, wherein the tubing adaptor is detachably fixed to the dental encasing component.

18. The oral appliance of claim 17, wherein the dental encasing component comprises at least one aperture in the anterior portion for accepting open projections of the tubing adaptor for attachment of the tubing adaptor, and for guiding through the dental encasing component.

19. The oral appliance of claim 15, wherein the tubing adaptor comprises at least one groove for detachably engaging the at least one tube when guiding air or oxygen from the at least one tube, through the tubing adaptor, and to the dental encasing component.

20. A tubing adaptor comprising:
one or more open projections for detachably engaging at least one aperture in an anterior portion of a dental encasing component, and for guiding air through the dental encasing component; and
at least one groove for detachably engaging at least one tube and guiding air or oxygen from the at least one tube, through the tubing adaptor, and to the dental encasing component.
